(19) United States
(12) Patent Application Publication
(10) Pub. No.: US 2019/0177696 A1
(43) Pub. Date: Jun. 13, 2019

(54) METHOD FOR PREPARING INDUCED HEPATIC PROGENITOR CELLS

(71) Applicant: ECOLE POLYTECHNIQUE FEDERALE DE LAUSANNE (EPFL), Lausanne (CH)

(72) Inventors: Carmen UNZU EZQUERRO, Geneve (CH); Marc FRIEDLI, Lausanne (CH); Didier TRONO, Vufflens-Chateau (CH); Barbara WILDHABER, Veyrier (CH)

(21) Appl. No.: 16/310,137

(22) PCT Filed: Jun. 16, 2017
(86) PCT No.: PCT/EP2017/064773

§ 371 Date: Dec. 14, 2018

(30) Foreign Application Priority Data
Jun. 16, 2016 (EP) 16174787.8

(51) Int. Cl. C12N 5/071

(52) U.S. Cl. CPC C12N 5/0672; C12N 2501/11; C12N 2506/14; C12N 2501/999; C12N 2501/115

(57) ABSTRACT

The present invention relates to a method for preparing induced hepatic progenitor cells (iHPC) comprising the steps of dedifferentiation of hepatocytes by culture with a culture medium comprising at least one activator of the Wnt signaling, Basic fibroblast growth factor (b-FGF), and Epidermal growth factor (EGF).

### FIELD OF THE INVENTION

The present invention is directed to methods for inducing the dedifferentiation of hepatocytes to induced hepatic progenitor cells (iHPC).

### BACKGROUND OF THE INVENTION

The liver has a unique regenerative capacity for an adult organ and both non-parenchymal cells and parenchymal hepatocytes contribute to regeneration. The repopulating capacity of these different liver populations has been proved in mouse models, demonstrating that the hepatocytes have a very active role in regeneration upon liver damage.

Nevertheless, human primary hepatocytes are quiescent cells that do not divide in vitro, remaining unclear if they maintain the innate proliferative ability upon isolation. This lack of division ex vivo is the main limitation for the progress of liver cell therapy. Transplantation of genetically corrected hepatocytes is an attractive alternative for many inherited metabolic liver diseases for which the only cure is orthotopic liver transplantation, a high-risk procedure also limited by shortage of donors.

Recently, landmark studies have demonstrated the possibility of changing cell fate by overexpressing the suitable transcription factors. Examples go from induced pluripotent stem cells (iPSC) reprogramming to direct trans-differentiation of fibroblasts to hepatocytes in vivo circumventing the pluripotent state. iPSC are endowed with intrinsic self-renewal ability and the potential to differentiate into any of the three germ layers. They allow cell amplification before differentiation, and if properly manipulated could constitute a large source of gene-corrected transplantable hepatocytes.

As such, iPSC are heralded as a most promising avenue for cell-based therapeutics. However, the generation of iPSC has the risk of inducing epigenetic abnormalities resulting notably in improper resetting of transposable elements control. This decreases the efficiency of reprogramming and re-differentiation of the cells and could potentially result in long-term complications, including oncogenic transformation after re-implantation.

Successful amplification of human primary bipotent biliary cells in 3D organoids and the expansion of adult-derived human liver mesenchymal-like cells have demonstrated that expansion of human liver cells is possible under the adequate culture conditions. Until very recently hepatocytes were not considered good candidates for amplification due to their quiescence in the absence of liver damage but different studies have also demonstrated direct induction of proliferation in human hepatocytes. Nevertheless, these techniques are based on cell transduction or resulted in low cell expansion.

There is thus an unfulfilled need for a novel, effective and efficient method to develop hepatic progenitor cells that can be proliferating in culture, to differentiate into hepatocytes and be used in therapy.

### SUMMARY OF THE INVENTION

The inventors met the burden to develop a method for generating proliferative hepatic progenitor cells from human hepatocytes by ex vivo pharmacological manipulation of the liver cells. Dedifferentiation was achieved by culturing them with specific growth factors and small molecules that mimic the Wnt and FGF signaling present during liver development in embryogenesis.

Thus, in a first aspect, the invention relates to a method for preparing induced hepatic progenitor cells (iHPC) comprising the step of dedifferentiation of hepatocytes by culture with a culture medium comprising at least one activator of the Wnt signaling, basic fibroblast growth factor (b-FGF) and epidermal growth factor (EGF).

In a second aspect, the invention pertains to a method for re-differentiating iHPC to hepatocytes, comprising the step of providing iHPC according of the invention and culturing the iHPC with adult primary hepatocytes.

In a third aspect, the invention relates to induced hepatic progenitor cells (iHPC) according to the invention, wherein said iHPC:
are capable of proliferating in a culture, and
are capable of differentiating into a hepatocytes or hepatic stellate cells (HSC).

In a fourth aspect, the invention relates to therapeutic uses of the induced hepatic progenitor cells or the hepatocytes obtained by the redifferentiation of said iHPC.

In a fifth aspect, the invention relates to the use of the induced hepatic progenitor cells or the hepatocytes obtained by the redifferentiation of said iHPC for the development of an artificial liver.

In a sixth aspect, the invention relates to a culture medium comprising at least one GSK-3 inhibitor, basic fibroblast growth factor (b-FGF) and epidermal growth factor (EGF), wherein culture medium is free of any expression system encoding OCT4, SOX2, KLF4 or any combination thereof or any cells that express OCT4, SOX2, KLF4 or any combination.

### DETAILED DESCRIPTION OF THE INVENTION

### Definition

As used herein, “induced hepatic progenitor cells, “Proliferative hepatic progenitor cells” or “iHPC” according to the invention refers to cells obtained by dedifferentiation of hepatocytes, preferably by culture with a culture medium comprising:
at least one activator of the Wnt signaling,
basic fibroblast growth factor (b-FGF), and
epidermal growth factor (EGF).
   Typically, iHPC according to the invention are positive for expression of the cell markers A1AT, KRT7, AFP, CXCR4, CD105, CD133, CD90, CD44 and C73. Typically, said iHPC are negative for expression of the cell markers ALB, CD34, LGR5, Oct4, Nanog or Sox2.

The term “reprogramming” as used herein refers to a process that alters or reverses the differentiation state of a differentiated cell.

The term “induced pluripotent stem cell” or “iPS cell” or “iPSC” refers to a type of pluripotent stem cell artificially derived from a non-pluripotent, adult somatic cell, by compulsory dedifferentiation (reprogramming). Typically, said iPS cells are reprogrammed by the expression of the transcription factors Oct4 (also known as POU5F1), Sox2, Klf4, and c-Myc (OKSM, or other variations of reprogramming cocktails).

The term “pluripotent” as used herein refers to a cell with the capacity, under different conditions, to differentiate to cell types characteristic of all three germ cell layers (endoderm, mesoderm and ectoderm). Pluripotent cells are characterized primarily by their ability to differentiate to all three germ layers, using, for example, an immuno-deficient mouse teratoma formation assay. A pluripotent cell is an undifferentiated cell.

The term “differentiated cell” refers to any primary cell that is not, in its native form, pluripotent as that term is defined herein. It is noteworthy that placing many primary cells in culture can lead to some loss of fully differentiated characteristics. Thus, simply culturing such cells is included in the term “differentiated cells” and does not render these cells non-differentiated cells (e.g. undifferentiated cells) or pluripotent cells. The transition of a differentiated cell to pluripotency requires a reprogramming stimulus beyond the stimuli that lead to partial loss of differentiated character in culture. Reprogrammed cells also have the characteristic of the capacity of self-renewal (extended passaging) without loss of growth potential, relative to primary parental cells, which generally have capacity for only a limited number of divisions in culture. In some embodiments, the term “differentiated cell” also refers to a cell of a more specialized cell type derived from a cell of a less specialized cell type (e.g. from an undifferentiated cell or a reprogrammed cell) where the cell has undergone a cellular differentiation process.

The term “dedifferentiation”” refers to a mechanism whereby differentiated cells regain properties of their ancestors, including stemness. This mechanism lead to the development of induced progenitor cells.

The term “redifferentiation” refers to respecialization of dedifferentiated cells.

### Method for Preparing Induced Hepatic Progenitor Cells

Until very recently, hepatocytes have been considered quiescent cells with a limited division rate that happened only upon liver damage for regeneration. For this reason and because of the difficultness of obtaining human liver samples, alternative sources of cells have been used to generate hepatocyte-like cells (HLC) for liver cell therapy. Despite the promising improvements in the differentiation protocols from pluripotent cells to generate HLC, the differentiation process is not completely effective and differentiated cells still show a fetal phenotype. Moreover, it has been shown that Induced pluripotent stem cells (iPSC) keep the 3D genome structure and the epigenetic memory of the cells of origin, which results in a differentiation bias.

The inventors have now developed a method for inducing hepatic progenitor cells in which hepatocytes are cultured together with a cocktail of growth factors and a small molecule. For this purpose, they have selected human primary hepatocytes as the cell source in order to minimize the impact on cell remodeling.

Therefore, in a first aspect, the invention relates to a method for preparing induced hepatic progenitor cells (iHPC) comprising the steps of dedifferentiation of hepatocytes by culture with a culture medium comprising, preferably consisting of, at least one activator of the Wnt signaling, Basic fibroblast growth factor (b-FGF) and Epidermal growth factor (EGF). Preferably, said method further comprises a step of expansion of iHPC.

Preferably, said hepatocytes are human, more preferably primary human hepatocytes. More preferably, said hepatocytes do not express OCT4, SOX2, KLF4 or any combination thereof.

Typically, said hepatocytes were isolated from patients undergoing transplantation. Dedifferentiation of the hepatocytes to proliferative induced hepatic progenitor cells (iHPC) is achieved between 5 and 7 days, preferably in less than 7 days. Said method allows the preparation of iHPC in less than 7 days. In these conditions cell expansion is exponential by 10⁴ to 10⁵ times.

Dedifferentiation of the cells was achieved by culturing them with specific growth factors and small molecules mimicking the signaling during liver development in embryogenesis. Without being bound by theory, the inventors believe that by inducing Wnt and FGF2 signaling, the expression of mature hepatocyte markers is downregulated and those of hepatic progenitors are upregulated, allowing the cells to start proliferation.

As used herein, the term “Wnt” is meant the family of highly conserved secreted signaling molecules which play key roles in both embryogenesis, tissue regeneration, and mature tissues. The human Wnt gene family has at least 19 members (Wnt-1, Wnt-2, Wnt-2B/Wnt-13, Wnt-3, Wnt3a, Wnt-4, Wnt 5A, Wnt-5B, Wnt-6, Wnt-7A, Wnt-7B, Wnt-8A, Wnt-8B, Wnt-9A/Wnt-14, Wnt-9B Wnt-15, Wnt-1 OA, Wnt-10B, Wnt-11, and Wnt-16). Wnt proteins modulate cell activity by binding to Wnt receptor complexes that include a polypeptide from the Frizzled (Fz) family of proteins and a polypeptide of the low-density lipoprotein receptor (LDLR)-related protein (LRP) family of proteins. Once activated by Wnt binding, the Wnt receptor complex will activate one or more intracellular signaling cascades. These include the canonical Wnt signaling pathway: the Wnt planar cell polarity (Wnt PCP) pathway: and the Wnt-calcium (Wnt/Ca2+) pathway.

As used herein, the term “activator of Wnt signaling pathway” or “activator of Wnt signaling pathway” refers to an agent that enhances or stimulates the normal functioning of Wnt signaling pathway, either by increasing transcription or translation of Wnt-encoding nucleic acid, and/or by inhibiting or blocking activity of a molecule that inhibits Wnt expression or Wnt activity, and/or by enhancing normal Wnt activity.

For example, the Wnt activator can be selected from an antibody, an antigen-binding fragment, an aptamer, an interfering RNA, a small molecule, a peptide, an antisense molecule, and another binding polypeptide.

In another example, the Wnt activator can be a polynucleotide selected from an aptamer, interfering RNA, or antisense molecule that interferes with the transcription and/or translation of a Wnt-inhibitory molecule.

Preferably, in the context of the invention, said Wnt activator is a small molecule. In a preferred embodiment, said activator of the Wnt signaling is selected from the group consisting of
inhibitors of the frizzled-related protein (SFRP) such as WAY-316606;
activators of protein phosphatase (PP2A) such as IQ 1;
activators of ADP-Ribosylation Factor GTPase Activating Protein 1 (ARFGAP1) such as QS11; and
inhibitors of GSK-3.

Alternatively, the activator of Wnt signaling is selected from the group consisting of Wnt3a, WNT 5, WNT-6a, FGF18, beta-catenin, norrin, R-spondin2, and a GSK-inhibitor.

In a more preferred embodiment, said activator of the Wnt signaling is an inhibitor of GSK3.

Glycogen synthase kinase 3 (GSK3) is a serine/threonine kinase that plays a key inhibitory role in both the insulin and Wnt signaling pathways.

A wide range of GSK3 inhibitors are known, by way of example, the inhibitors CHIR99021, CHIR98014, AR-AO144-18, TDZD-8, SB216763 and SB415286. Non limiting examples of GSK-3 inhibitors are detailed in WO03/004472, WO03/004475 and WO03/089419. Preferably, the GSK-3 inhibitor is CHIR99021.

CHIR99021, also called CT 99021, is an aminopyrimidine derivative that inhibits GSK3α and GSK3β with IC50 values of 10 and 6.7 nM, respectively.

CHIR99021 has also been shown to induce the reprogramming of murine and human somatic cells into stem cells. The chemical name of CHIR99021 is 6-[[2-[[4-(2,4-Dichlorophenyl)-5-(5-methyl-1H-imidazol-2-yl)-2-pyrimidinyl]amino]ethyl]amino]-3-pyridinecarbonitrile. Its formula is C₂₂H₁₈Cl₂N₈ and its molecular weight is of 465.3 g/mol. Finally, the CAS number of CHIR99021 is 252917-06-9.

Basic fibroblast growth factor (b-FGF) is a potent angiogenic factor and endothelial cell mitogen.

Epidermal growth factor (EGF) is a growth factor that stimulates cell growth, proliferation, and differentiation by binding to its receptor EGFR. EGF induces hepatocyte division.

In a preferred embodiment, the step of dedifferentiation of hepatocytes is performed with a culture medium comprising
CHIR99021 at a concentration comprised between 0.1 μM to 50 μM, preferably between 1 μM to 10 μM, preferably between 1 μM and 5 μM, more preferably about 3 μM;
EGF at a concentration comprised between 0.5 ng/ml to 120 ng/ml, preferably between 1 ng/ml to 50 ng/ml, preferably between 5 ng/ml and 20 ng/ml, more preferably about 10 ng/ml; and
FGF at a concentration comprised between 0.5 ng/ml to 120 ng/ml, preferably between 1 ng/ml to 50 ng/ml, preferably between 5 ng/ml and 20 ng/ml, more preferably about 10 ng/ml.

In a preferred embodiment, said culture medium further comprises a compound selected from the group consisting of BMP4, VEGF, HGF, TGF-beta, dilauroyl phosphatidylcholine (DLPC), A83 (transforming growth factor-b type I receptor inhibitor), Activin A, a DNA methyltransferase inhibitor, Parnate, and Sodium butyrate (NaB).

The culture medium of the invention may comprise a basal medium. The expression “basal medium” refers to a medium that supplies essential sources of carbon and/or vitamins and/or minerals for the cells. The basal medium is generally free of protein and incapable on its own of supporting self-renewal of cells. The iron transporter provides a source of iron or provides the ability to take up iron from the culture medium. Suitable iron transporters include transferrin and apotransferrin. It is preferred that the medium further comprises one or more of insulin or insulin-like growth factor and albumin (preferably recombinant) or albumin substitute, and is free of feeder cells and feeder cell extract. The medium may also comprise an inhibitor of apoptosis or any other component that promotes the maintenance of pluripotent cells in culture.

In one embodiment, said culture medium is free of any expression system encoding OCT4, SOX2, KLF4 or any combination thereof. Preferably, said culture medium is free of any cells that express OCT4, SOX2, KLF4 or any combination thereof.

Preferably, the step of expansion of iHPC of the method of the invention lasts from 20 to 60 days. In a preferred embodiment, the iHPC of the invention express the proliferation marker KI67.

Method for Re-Differentiating iHPC

In a second aspect, the invention relates to a method for re-differentiating iHPC to hepatocytes, comprising the step of providing iHPC according to the method of preparing iHPC as described herein and culturing said iHPC with adult primary hepatocytes.

The inventors have shown that the iHPC have the ability to proliferate in a culture, and to differentiate into hepatocytes or hepatic stellate cells (HSC).

Further, the inventors have shown the role of the surrounding extracellular matrix, whether said matrix is collagen or matrigel. Typically, the proportion of HSC was found to be higher in matrigel than in collagen. Therefore, the person skilled in the art would know how to adapt the nature of the surrounding extracellular matrix, depending on the targeted results and the targeted uses of the generated hepatocytes or HSC.

### Induced Hepatic Progenitor Cells

In a third aspect, the invention relates to induced hepatic progenitor cells (iHPC) obtained by the dedifferentiation of hepatocytes by culture in a culture medium comprising at least one GSK-3 inhibitor, basic fibroblast growth factor (b-FGF) and epidermal growth factor (EGF), wherein said iHPC:
are capable of proliferating in a culture, and
are capable of differentiating into hepatocytes or hepatic stellate cells (HSC).

The inventors have carried a thorough analysis of the cell markers' expression of iHPC. Typically, the iHPC according to the invention are positive for expression of alpha-1-antitrypsin (A1AT), Hepatocyte nuclear factor 4 alpha (HNF4a), Cytokeratin 7 (KT7), Alpha fetoprotein (AFP), CD73, CD90, CD105, CD133, CD44, and C-X-C chemokine receptor type 4 (CXCR4).

Interestingly, the inventors have further shown that the iHPC and iPSC differ from the cell markers expression. Thus, the invention also relates to induced hepatic progenitor cells (iHPC) which are positive for expression of the cell surface markers CD105 and CD73.

Typically, said iHPC are negative for expression of the cell surface markers Oct4, Nanog or Sox2.

Typically, said iHPC are negative for expression of albumin.

### Therapeutic Uses of the Induced Hepatic Progenitor Cells and the Redifferentiated Hepatocytes

In a fourth aspect, the invention relates to therapeutic uses of the induced hepatic progenitor cells and the hepatocytes obtained by the redifferentiation of said iHPC.

The invention has a number of therapeutic applications. The iHPC and the the hepatocytes obtained by the redifferentiation of said iHPC of the invention constitute a highly promising strategy for providing new sources of cells to replace a damaged liver to all patients who require transplantation.

In a specific embodiment, the invention relates to the induced hepatic progenitor cells or the hepatocytes obtained by the redifferentiation of said iHPC for use for liver cell therapy. The iHPC and the hepatocytes obtained by the redifferentiation of said iHPC thus constitute a potential therapy for patients with life-threatening liver diseases while excluding any tumorigenic potential. Thus, in a further embodiment, the invention relates to the induced hepatic progenitor cells or the hepatocytes obtained by the redifferentiation of said iHPC for use for the treatment of a hepatic disease. Preferably, said hepatic disease is selected from the group consisting of cirrhosis, acute liver failure, inborn errors of metabolism, hepatitis, liver cancer, alcoholic liver disease, hepatic steatosis, liver fibrosis, liver cysts, hemochromatosis, alcoholic hepatitis, Wilson's disease, Gilbert's syndrome, jaundice, liver hemangioma, non-alcoholic fatty liver disease, and nonalcoholic steatohepatitis,

Said therapy may be in combination with gene therapy, drug screen, or drug testing.

### Use of the Induced Hepatic Progenitor Cells for the Development of an Artificial Liver

In a fifth embodiment, the invention relates to the use of induced hepatic progenitors or the hepatocytes obtained by the redifferentiation of said iHPC for the development of an artificial liver.

Said artificial liver may constitute a promising strategy for therapy or drug testing.

### Culture Medium

In a sixth aspect, the invention relates to a culture medium comprising at least one activator of the Wnt signaling, basic fibroblast growth factor (b-FGF) and epidermal growth factor (EGF). In one embodiment, said culture medium is free of any expression system encoding OCT4, SOX2, KLF4 or any combination thereof. Preferably, said culture medium is free of any cells that express OCT4, SOX2, KLF4 or any combination thereof.

The above mentioned technical features are applicable here.

Preferably, the invention relates to a culture medium consisting of a GSK-3 inhibitor, basic fibroblast growth factor (b-FGF) and epidermal growth factor (EGF).

Particularly preferred media according to the invention comprise:
CHIR99021 at a concentration comprised between 0.1 μM to 50 μM; preferably between 1 μM to 10 μM, preferably between 1 μM and 5 μM, more preferably about 3 μM;
EGF at a concentration comprised between 0.5 ng/ml to 120 ng/ml; preferably between 1 ng/ml to 50 ng/ml, preferably between 5 ng/ml and 20 ng/ml, more preferably about 10 ng/ml and
FGF at a concentration comprised between 0.5 ng/ml to 120 ng/ml, preferably between 1 ng/ml to 50 ng/ml, preferably between 5 ng/ml and 20 ng/ml, more preferably about 10 ng/ml.

The present invention enables the preparation of iHSC and improved culture of iHSC in medium that is preferably free of serum, serum extract, feeder cell and feeder cell extract.

The invention will be further illustrated by the following examples. However, these examples should not be interpreted in any way as limiting the scope of the present invention.

### EXAMPLE

Material and Methods

Cell culture. Primary human hepatocytes were isolated by 3 step liver perfusion from surgical resection pieces of children undergoing total hepatectomy and treated in the Swiss Center for Liver Diseases in Children at the University Hospitals of Geneva (after parents' written consent and approval from the Canton of Geneva Ethics committee), as described in Birraux, J., et al., A step toward liver gene therapy: efficient correction of the genetic defect of hepatocytes isolated from a patient with Crigler-Najjar syndrome type 1 with lentiviral vectors. Transplantation, 2009. 87(7): p. 1006-12. Briefly, 2×10⁵ human primary hepatocytes from all donors were plated on collagen-coated wells and maintained in Hepatocyte Culture Medium (HCM Bullet kit. Lonza, Basel, Switzerland).

Induced hepatic progenitor cells (iHPC) were generated by culturing the human primary hepatocytes in DMEMF12 Ham 15 mM hepes with Na bicarbonate (Sigma) 1% glutamine, 1% Penicillin/Streptomycin, 1% non-essential aminoacids, 10% Knock out Serum replacer (Gibco), 5% FBS, 10 ng ml⁻¹ Epidermal Growth Factor (EGF) (Peprotech), 10 ng ml⁻¹ basic fibroblast growth factor (bFGF) (R&D) and 3 uM CHIR99021 (SIGMA). Cells were cultured on collagen-coated plates and splitted with StemPro accutase (Gibco).

iPSC generation. The polycistronic excisable reprogramming vector STEMCCA (hereafter called OKSM) was kindly provided by Prof. G. Mostoslaysky (Boston University, MA, USA). Lentiviral particles encoding Oct-4, Klf4, Sox2 and c-Myc were prepared as previously described in Birraux, J., et al. Transplantation, 2009. 87(7): p. 1006-12.

5×10⁵ human primary hepatocytes were plated on Matrigel or collagen before being transduced with OKSM using a MOI of 20. After 5 days, cells were switched to mTeSR1 medium (Stemcell Technologies) and grown until reprogrammed colonies emerged (˜20 days). After 5 days, cells were switched to mTeSR1 medium and grown on a mouse fibroblast feeder layer until reprogrammed colonies emerged (˜21 d). Individual human iPSC clones were then picked and expanded on matrigel-coated plates in mTeSR1 medium.

iPSC characterization. Expression of pluripotency markers was addressed by immunofluorescence. iPSC were plated on matrigel-coated glass coverslips. Before staining, coverslips were rinsed once with PBS and fixed for 15 minutes with 4% paraformaldehyde (PFA). Cells were then rinsed 3 times with PBS for 5 minutes and blocked-permeabilized with 3% bovine serum albumin (BSA) with 0.3% Triton X-100 in PBS for 1 h. Primary antibodies at appropriate dilutions were incubated ON at 37° C. in PBS containing 0.1% Tween-20 and 1% BSA. Secondary antibodies at appropriate dilutions were incubated for 1 hour at 37° C. together with DAPI 1:400. Finally, cells were washed and mounted onto glass slides with Mowiol and left overnight in the dark at room temperature (RT) before observation under the microscope. The kit of primary antibodies Stemlight pluripotency kit (Cellsignaling technology), which includes antibodies against Oct4, Sox2, Nanog, SSEA4 and Tra-1-60 was used.

Secondary antibodies were donkey anti-mouse alexa fluor 488 and donkey anti-rabbit alexa fluor 536 (Life technologies). Images were visualized with a Zeiss axiophoto microscope (Carl Zeiss) equipped with an Axiocam camera (Carl Zeiss); confocal images were obtained using the LSM 510 laser scanning confocal microscope (Carl Zeiss). Embryoid bodies from the different clones were generated using the Agrewell plates and following manufacturers' instructions (STEMCELL technologies). Karyotype analysis in all the iPSC clones following the G-banding method was performed by the service of Cytogenetics of the University of Geneva. 1×10⁶ cells from the different iPSC clones at passage 3 in 100 μl of matrigel-PBS mix (1:1) were injected in a subcutaneous or intratesticular location in NOD/SCID mice to induce teratoma formation. hES were injected as a control. Three animals were injected per clone. Mice were sacrificed after 3 to 8 weeks, according to tumor size and development. Tumors were fixed in 10% buffered formalin for 48 h.

Lentiviral Vectors. The vector encoding Green Fluorescent Protein under the control of the transthyretin promoter. The luciferase vector under the control of the PGK promoter was produced by GEG Tech (France). Cells were transduced at an MOI of 40.

Immunofluorescence and antibodies. For immunofluorescence, cells were cultured on slides and fixed for 15 minutes with 4% paraformaldehyde (PFA). Cells were then rinsed 3 times with PBS for 5 minutes and blocked-permeabilized with 3% bovine serum albumin (BSA) with 0.3% Triton X-100 in PBS for 1 h. Primary antibodies at appropriate dilutions were incubated ON at 37° C. in PBS containing 0.1% Tween-20 and 1% BSA. Secondary antibodies at appropriate dilutions were incubated for 1 hour at 37° C. together with DAPI 1:400. Finally, cells were washed and mounted onto glass slides with Mowiol.

Antibodies, flow cytometry, cell sorting. Intracellular stainings were done with the Cytofix/Cytoperm kit (BD). For cell sorting, primary hepatocytes and iHPC were incubated with Hoechst33342 (Invitrogen) at 15 ug/ml together with reserpine (Sigma) at 5 uM for 30 min at 37° C. Cells were either acquired on a Gallios or sorted using a FACSAria II (BD), and analyzed using FlowJo (Tree Star) or Kaluza softwares.

DNA sequencing and human cytoSNP-12 DNA array. Genomic DNA of harvested cells was isolated using the QIAmp DNA Mini-kit (Qiagen). Crigler-Najjar type I mutation was detected by sequencing an amplicon of 295 bp in the exon 4 region. 200 ng of DNA were loaded in the human cytoSNP-12 DNA Bead Chip (Illumina) in order to detect genetic and structural variations. The array includes 220,000 markers for cytogenetic analysis covering around 250 genomic regions.

Protein expression. Albumin secretion was measured in the culture medium using the ELISA kit for human Albumin from ICL according to the manufacturer's instructions. Cytochrome CYP3A4 activity was detected by luminescence using the P450-Glo CYP3A4 kit from Promega.

Mice strains and cell transplantation. Experimental protocols were performed according to European Council Guidelines and the Swiss Federal Veterinary Office. Acceptable standards of human animal care and treatment employed in these mice and the experimental design of this study were approved by the Ethics Committee for Animal Care of the Vaud Region in Switzerland (license VD2865). For iHPC transplantation, NOD-Cg-Prkdcscid I12rgtmlWjl/SzJ (NSG) mice were pretreated with retrorsine (2 intraperitoneal injections 4 and 2 weeks before transplantation, 70 mg/kg).

In vivo luciferase measurements. Non-invasive luciferase expression measurements were performed in living mice 7 days after cell transplantation. 100 μl of d-luciferin (30 mg/ml in 150 mM NaCl) were injected intraperitoneally and mice were anesthetized with isoflorane during the imaging. Each mouse was placed in the imaging chamber of a Xenogen IVIS system (Xenogen, Alameda, Calif.), which includes a cooled charge-coupled device (CCD) camera. A gray-scale photograph of the animals was acquired, followed by bioluminescence image acquisition (2 min). Regions of interest (ROIs) were traced over the positions of greatest signal intensity on the transplanted and control animals, which were used as background readings. Light intensity was quantified as photons/second/cm²/sr. The gray-scale photograph and data images from all studies were superimposed, using Living Image software (Xenogen).

RNA isolation, sequencing and expression. Total RNA was extracted either with TRIzol Reagent (Life Technologies), purified using the miRNeasy kit (Qiagen) and treated with RNase-Free DNase (Qiagen), or extracted with the NucleoSpin RNA kit (Macherey-Nagel) with an on column DNase treatment. RNAs were reverse-transcribed using random hexamers and SuperScript II (Invitrogen). Sample libraries were prepared using a TruSeq RNA sample preparation kit (Illumina). Libraries were sequenced with 100-base single or paired-end reads on an Illumina Hi-Seq machine.

Bioinformatic analyses. Heatmaps. Gene expression profiles were downloaded from the FANTOM5 CAGE dataset [54]. Clustering was computed using complete method and pearson distances for both row and columns.

Results

Hepatocyte Plasticity and Induction of Hepatic Progenitor Cells

Reprogramming of cells to induced pluripotent stem cells (iPSC) is a process of complex mechanisms which efficiency has been described to be dependent on cell division rate. Despite their lack of division, overexpression of the 4 classic pluripotency transcription factors (Oct4, Klf4, Sox2 and c-Myc, OKSM) in primary hepatocytes from a Crigler-Najjar patient resulted in cell reprogramming to iPSC.

Successful reprogramming of hepatocytes suggests that these cells still keep their intrinsic plasticity in vitro but they need an external boost for proliferation, such as MYC expression, since transduction of hepatocytes with an OKS vector did not succeed.

Following the same rationale of cell reprogramming to pluripotency, the inventors next investigated if the combination of Wnt and FGF signaling was able to dedifferentiate human hepatocytes to expandable hepatic progenitors. To test this hypothesis, primary hepatocytes isolated from six livers of pediatric patients undergoing transplantation were cultured together with a cocktail of growth factors and a small molecule:
The GSK-3 inhibitor CHIR99021, that induces Wnt signaling and is also used for chemical reprogramming of cells,
b-FGF, and
EGF, which induces hepatocyte division.

Dedifferentiation of the hepatocytes to proliferative induced hepatic progenitor cells (iHPC) was achieved in less than 7 days. CFSE staining for addressing cell proliferation in hepatocytes confirmed the induction of proliferation. More than 90% of the generated iHPC expressed the proliferation marker KI67.

FACS analysis of primary hepatocytes and iHPC revealed two different populations of cells according to their size and complexity in both subsets. DNA Hoechst staining identified one of these populations as diploid (2n) cells whereas the other population grouped multinucleated tetraploid (4n) and octaploid (8n) hepatocytes. Co-staining of the definitive endoderm marker CXCR4 and KI67 showed that 2n hepatocytes became both CXCR4 and KI67 positive upon iHPC generation. 4n and 8n cells were also dividing but remained negative for CXCR4. After several passages, the inventors detected a high enrichment of 2n iHPC but 4n and 8n cells were still present. In these conditions cell expansion was exponential by 104-5 times.

Induced Hepatic Progenitor Cells Marker Expression and Differentiation Properties

In culture, iHPC expressed a combination of endoderm hepatic progenitor and mesenchymal stem cell markers.

The results are summarized in the following table:

| | | Hepatocytes | iHPC |
|---|---|---|---|
| Albumin | ALB | + | − |
| cytochrome p450 3A4 | CYP3A4 | + | − |
| Alpha-1-antitrypsin | A1AT | + | + |
| Hepatocyte nuclear factor 4 alpha | HNF4a | + | /+ |
| Cytokeratin 7 | KT7 | − | + |
| Alpha fetoprotein | AFP | − | + |
| CD73 | CD73 | − | + |
| CD90 | CD90 | − | + |
| CD105 | CD105 | − | + |
| CD133 | CD133 | − | + |
| CD44 | CD44 | /+ | + |
| C—X—C chemokine receptor type | CXCR4 | − | + |
| 4 | | | |
| CD34 | CD34 | − | − |
| CD31 | CD31 | − | − |
| Leucine-rich repeat-containing | LGR5 | − | − |
| G-protein coupled receptor 5 | | | |
| alpha-smooth muscle actin | a-SMA | − | − |

In this table:
+ EXPRESSED
− NOT EXPRESSED
/+ PARTIALLY EXPRESSED

Expression of the mature hepatocyte markers albumin and HNF4a was lost during the first week, whereas expression of alpha-1-antitrypsin (A1AT) was kept in iHPC. Moreover iHPC generation induced the expression of the hepatoblasts markers KRT7 and AFP in the cells. CD73, CD90, CD105 and CD133 mesenchymal stem cell markers were also upregulated in iHPC independently of the ploidy of the cells. Interestingly, the inventors could detect differential expression between 2n and 4n/8n cells of CD44, another mesenchymal stem cell marker.

To rule out the presence of non-parenchymal or hematopoietic cells in the hepatocyte isolation, the inventors checked CD31 endothelial marker, LGR5 bipotent biliary cells marker and CD34 hematopoietic marker expression. The inventors didn't detect expression of any of these markers in primary hepatocytes and CD34 and LGR5 expression did not change in iHPC. However, CD31 endothelial marker was slightly upregulated in iHPC. This heterogeneity of marker expression mimics the level of complexity in liver embryogenesis, with different levels of mesenchymal and endoderm hepatic progenitors.

Given the dual expression of endoderm and mesenchymal markers the inventors wanted to address the differentiation ability of iHPC depending on the surrounding extracellular matrix. Hepatospheres of iHPC-ttr-GFP and primary hepatocytes were generated in PEG microwells and two different matrices were tested, collagen and matrigel. In both cases, differentiation of the cells was achieved by day 5. In the collagen condition, most of the iHPC spontaneously differentiated into hepatocytes. Nevertheless, iHPC also differentiated to hepatic stellate cells (HSC), detected by their characteristic morphology and aSMA staining demonstrating the bipotency of these progenitors. The proportion of HSC was higher in matrigel than in collagen highlighting the importance of the extracellular signaling for determining cell fate.

Transcriptome Comparison of iHPC Generation to iPSC Reprogramming from Hepatocytes

Next, the inventors performed RNA sequencing on iHPC and compared their transcriptome to the one from iPSC generated from the same donor and to the primary hepatocytes of origin. Re-differentiated hepatocytes from iHPC and hepatocyte-like cells differentiated (HLC) from iPSC were also included to address and compare the cell remodeling undergoing at the transcriptome level during both processes. For iHPC de-differentiation, cells from Crigler-Najjar (CNI) and Citrulinemia (CIT) patients were sequenced troughout the processes. For iPSC reprogramming and differentiation, RNA sequencing was performed in two different clones obtained from CNI patient specific hepatocytes.

For differentiation, iHPC were cultured in 20 ng ml⁻¹ Oncostatin M+1 μM Dexamethasone for 7 days. The re-differentiated cells increased the expression of mature hepatocyte markers such as albumin and HNF4a that were undetectable in the iHPC. A1AT and GATA4 expression also increased whereas the endoderm and hepatoblasts markers AFP, CXCR4 and KRT expression decreased during the differentiation. Hepatocyte-like cells (HLC) were differentiated from iPSC following the protocol described by Si-Tayeb et al.

Principal component analysis (PCA) showed a first clustering of the samples according to their level of differentiation during both processes. The heatmap of the 5000 most differentially expressed protein-coding genes showed clearly this clustering by the differentiation level. Of note, PCA1 vs. PCA3 showed that dedifferentiation of hepatocytes to iHPC and its re-differentiation towards hepatocytes is a more straight process compared to reprogramming and iPSC differentiation to HLC. Nevertheless, in both situations differentiated cells kept on expressing transcripts from earlier stages besides mature hepatocyte transcripts.

To address these similarities and differences further, the inventors performed a detailed analysis of cell-stage specific markers. As expected, transduction of hepatocytes with the OKSM lentiviral vector resulted in the induction of the expression of stem cell markers that leaded to the reprogramming of the cells to iPSC. During this process silencing of specific hepatic transcription factors and genes happened.

In the case of iHPC generation, KLF4, CD133 and the endoderm stem cell marker KDR were induced but not Oct4, Nanog or Sox2. Induction of KDR expression happened also during differentiation of iPSC to HLC. Interestingly, the inventors observed an overexpression of the mesenchymal markers CD105, C73 and CD90 during the reprogramming that was not maintained in the iPSC, with the exception of CD90. Expression of endoderm and hepatoblasts specific markers and transcription factors was maintained during hepatocyte dedifferentiation like some markers from mature hepatocytes such as A1AT. However, expression levels of AFP were low in iHPC compared to HLC.

Differentiation of iPSC towards HLC decreased the total percentage of differentially expressed transcripts when compared to primary HEP. Nevertheless this percentage was higher in HLC than in iHPC suggesting that dedifferentiation of primary hepatocytes involves more subtle cell remodeling compared to iPSC reprogramming.

Transposable Elements Expression During Cell Remodeling

Transposable elements (TE) represent over half of the human genome and transcript expression from these elements has demonstrated to be a specific tool for distinguishing between naïve and primed pluripotent cells (Theunissen and Friedli, Cell Stem Cell. 2016). Besides, it has been reported that iPSC reprogramming has heterogeneous effects on the regulation of these elements. Therefore, the inventors wanted to address how the expression of TEs (transposcriptome) changes during hepatocyte dedifferentiation to iHPC.

The heatmap of the 10000 most differentially expressed TE transcripts, showed that expression was cell stage specific following a similar pattern to protein-coding heatmaps. However, in this case differences in expression are highlighted compared to the protein-coding genes. When the inventors looked at specific TE family expression throughout both iHPC generation and iPSC reprogramming they detected an upregulation of the ERV family in both processes. This was expected for iPSC, where the expression of the HERVH integrant of the ERV family has been described as essential for the embryonic stem cell identity. Interestingly, the inventors also observed an up-regulation of the ERV family when dedifferentiation to iHPC was induced. Specific TE integrant analysis revealed that HERVH was also over expressed compared to hepatocytes when iHPC are generated as in iPSC reprogramming. On the other hand, the inventors did not detect a significant upregulation of any specific integrant in primary hepatocytes compared to both iHPC or iPSC. Importantly, 80% of the HERVH integrants expressed in iHPC overlapped with the integrants expressed in iPSC confirming the stemness of these cells and suggesting that TE expression analysis could be a fine-tuned barcoding for detecting different cell subsets such as cell progenitors.

To rule out that the differences between the same samples in the protein-coding transcriptome and the transposcriptome were due to DNA rearrangements or mutations, the inventors performed a copy number variation (CNV) array. They could not detect any consequence at this level when they induced the dedifferentiation of hepatocytes to iHPCs. However, reprogramming to iPSC caused a deletion in the clone that was undetectable by karyotype analysis.

iHPC Engraftment and Re-Differentiation In Vivo

In order to confirm the re-differentiation of iHPC also in vivo, the inventors transplanted the cells into the liver of immunodeficient NSG mice by intrahepatic injection. Animals were preconditioned with retrorsine and partial hepatectomy to ensure engraftment of the cells. Cells were injected in the remaining caudale lobe. For in vivo detection, iHPC were previously transduced with a lentiviral vector encoding the reporter transgene luciferase (LV-PGK-LUC). Engraftment of iHPC was confirmed by luminescence. Next, iHPC were transduced with a lentiviral vector encoding GFP under the control of the hepatospecific transthyretin (TTR) promoter in order to address maturation of the cells. To obtain a detailed imaging of the engrafted cells, the injected lobe was fixed and cleared out of lipids. The result was a near transparent piece of libver. GFP positive cells were detected throughout the injected region. Immunostaining in frozen sections of the injected liver lobes allowed the inventors to detect co-expression of GFP and the mature hepatocyte markers A1AT and cytochrome CYP3A4. GFP positive hepatocytes were also detected by immunohistochemistry. Therefore, the inventors were able to confirm that iHPC not only engrafted into the liver but also differentiated into mature hepatocytes demonstrating their potential for liver cell therapy.

### CONCLUSION

As progenitors, iHPC showed a limited growth that was reduced when the factor cocktail was removed from the medium or differentiation of the cells was induced. The inventors never detected tumor formation in vivo when the cells were injected in mice subcutaneously, intravenously or intrahepatic. Importantly, the six donors of hepatocytes were paediatric patients that underwent liver transplantation.

In vivo they found that the cells differentiated to hepatocytes. In order to address the potential of iHPC for liver gene and cell therapy combination, hepatocytes were transduced with the lentiviral vector encoding the GFP reporter transgene under the control of the transthyretin (ttr, pre-albumin) hepatospecific promoter. Upon transduction, more than 90% of hepatocytes expressed GFP and transgene expression was maintained during induction and in proliferative iHPC confirming their hepatoblast profile. Given that an overexpression of 5% of the missing protein has been reported to be enough to revert the phenotype in different homozygous disease models, iHPC provide a very promising methodological lead for the treatment of inborn hepatic diseases.

1. A method for preparing induced hepatic progenitor cells (iHPC) comprising the steps of dedifferentiation of hepatocytes by culture with a culture medium comprising
at least one activator of the Wnt signaling,
Basic fibroblast growth factor (b-FGF), and
Epidermal growth factor (EGF).

2. The method according to claim 1, wherein said activator of the Wnt signaling is a GSK-3 inhibitor.

3. The method according to claim 2, wherein said GSK-3 inhibitor is CHIR99021.

4. The method according to claim 3, wherein
CHIR99021 is present in said culture medium at a concentration comprised between 0.1 μM to 50 μM;
EGF is comprised in said culture medium at a concentration comprised between 0.5 ng/ml to 120 ng/ml; and
FGF is comprised in said culture medium at a concentration comprised between 0.5 ng/ml to 120 ng/ml.

5. The method according to claim 1, wherein said culture medium further comprises a compound selected from the group consisting of BMP4, VEGF, HGF, TGF-beta, dilauroyl phosphatidylcholine (DLPC), A83 (transforming growth factor-b type I receptor inhibitor), Activin A, a DNA methyltransferase inhibitor, Parnate, and Sodium butyrate (NaB).

6. The method according to claim 1, wherein
said hepatocytes are primary human hepatocytes that do not express OCT4, SOX2, KLF4, and
said culture medium is free of any expression system encoding OCT4, SOX2, KLF4 or any combination thereof.

7.-15. (canceled)
